(19) United States
(12) Patent Application Publication
(10) Pub. No.: US 2024/0058592 A1
(43) Pub. Date: Feb. 22, 2024

(54) PATENT DUCTUS ARTERIOSUS SEVERITY SCORES TO PREDICT CLINICAL OUTCOMES

(71) Applicants: Rush University Medical Center, Chicago, IL (US); UH Rainbow Babies Children's Hospital, Cleveland, OH (US)

(72) Inventors: John BOKOWSKI, Chicago, IL (US); Krishna Kishore UMAPATHI, Chicago, IL (US); Aravind THAVAMANI, Cleveland, OH (US)

(73) Assignees: Rush University Medical Center, Chicago, IL (US); UH Rainbow Babies Children's Hospital, Cleveland, OH (US)

(21) Appl. No.: 18/133,488

(22) Filed: Apr. 11, 2023

Related U.S. Application Data
(60) Provisional application No. 63/399,107, filed on Aug. 18, 2022.

(51) Int. Cl. A61M 60/178; A61M 60/515

(52) U.S. Cl. CPC A61M 60/178; A61M 60/515; A61M 2230/04

(57) ABSTRACT

The present disclosure is directed to methods of utilizing echocardiography for assessing hemodynamic significance of patent ductus arteriosus (PDA) at early time points to supplement a patient's clinical and laboratory data by determining a PDA severity score. The present disclosure is further directed to treating the patient for PDA based on the severity score.

### CROSS-REFERENCE TO RELATED APPLICATION

This application claims the benefit of U.S. Provisional Application No. 63/399,107, filed Aug. 18, 2022, which is incorporated by reference herein.

### BACKGROUND

Patent ductus arteriosus (PDA) is a congenital heart defect in the early life of a preterm infant in which an extra blood vessel, or the ductus arteriosus, is part of a baby's blood flow system in the womb leading from the heart. It allows blood to skip the circulation to the lungs. In normal babies, PDA closes on its own shortly after birth. PDA that stays open longer, may cause extra blood to flow to the lungs, putting pressure on the heart and lungs. Thus, PDA may be physiologic, sometimes beneficial (e.g., during pulmonary hypertension) or pathologic (e.g., pulmonary congestion and/or systemic under perfusion).

It is difficult to assess the hemodynamic aspects of PDA merely by evidence of clinical signs. Thus, widespread ambiguity exists about hemodynamic significance in a preterm infant with PDA (Evans N., (2005), incorporated by reference herein with respect to such background teaching). Many randomized control trials have found that treatment of PDA does not impact mid-term and long-term outcomes associated with PDA. These outcomes may include intraventricular hemorrhage (IVH), chronic lung disease (CLD), necrotizing enterocolitis (NEC), neurobehavioral problems, and mortality (Cooke, (2003); El-Khuffash, (2010); Cunha, (2005); Shortlan, (1990); Brooks, (2005); El-Khuffash, (2007); Schmidt, (2006); Benitz, (2010), incorporated by reference herein with regard to such background teaching). However, randomized control trials have failed to demonstrate any causal link between PDA and the above mentioned outcomes (Schmidt, (2006); Shah, (2006); Evans, (2003); Ohlsson, (2003), incorporated by reference herein with regard to such background teaching). This may be because these studies have failed to evaluate the early hemodynamic impact of PDA before initiating treatment.

PDA may lead to systemic under perfusion and pulmonary over circulation when a shunt volume across the PDA is high. However, no demonstrated direct echocardiographic markers related to the shunt volume across PDA have been identified. Surrogates or indicators of PDA shunt volume have been used to assess presence of pulmonary over circulation. For example, PDA diameter, left atrial size and aortic root diameter ratio (LA:Ao ratio), and/or systemic under perfusion (e.g., decreased flow in celiac artery and superior mesenteric artery, percent (%) of diastolic flow reversal in the descending aorta) have been used to assess the presence of pulmonary over circulation. However, significant heterogeneity exists amongst studies with overt importance on PDA diameter (Bussmann, (2018); Zonnenberg, (2011), incorporated by reference with regard to such background teaching).

There remains a need for robust techniques to measure reliable variables that may be predictive of PDA, help in determining the flow dynamics of a PDA shunt, and that may be essential to inform early decision-making and assessing the need for possible pharmacological treatment or device closure. Potential benefits of using an echocardiogram to assess PDA includes defining the role PDA plays in the development of important clinical outcomes, thereby helping determine the need for early treatment (El-Kuffash, (2011) incorporated by reference with regard to such background teaching).

### SUMMARY

One aspect of the present disclosure is directed to a method of treating Patent Ductus Arteriosus (PDA) in a subject comprising identifying a clinical variable of the subject; measuring one or more echocardiographic variables of the subject one week after birth; determining a PDA severity score using a regression model; and treating PDA in the subject comprising administering an effective amount of a pharmacological agent or performing a surgical procedure; wherein the severity score is above a threshold PDA severity score (PDAss).

In some embodiments, the clinical variable and echocardiographic variable may be a measurement associated with PDA in the subject. In some embodiments, the clinical variable may comprise a gestational age. In some embodiments, the echocardiographic variable measurement may comprise a level of systemic perfusion, a level of pulmonary perfusion, or a left ventricular LV function. In some embodiments, the echocardiographic variable measurement may be a pulmonary perfusion index. In some embodiments, the echocardiographic variable measurement may be a left ventricle output. In some embodiments, the echocardiographic variable measurement may be a superior mesenteric artery velocity time integral. In some embodiments, the echocardiographic variable measurement may be a pulmonary vein diastolic flow. In some embodiments, the echocardiographic variable measurement may be a reversal of flow in diastole in descending aorta.

In some embodiments, the subject may comprise a neonate having a gestational age less than about 32 weeks. In some embodiments, the regression model is:

PDAss=(Gestational Age×−0.359)+(PPI in L/min/m²×0.418)+(LVO in mL/kg/min×0.001)+(SMA VTI in cm×−0.041)+(PV Vd in m/s×0.434)+(DFR in %×0.150)+11 (constant)

wherein PPI is a pulmonary perfusion index measurement, LVO is a left ventricle measurement, SMA VTI is a superior mesenteric artery velocity time integral, PV Vd is a pulmonary vein diastolic flow measurement, and DFR is a reversal of flow in diastole in descending aorta measurement of the subject.

In some embodiments, the regression model is a multivariate logistic regression model. In some embodiments, the PDA severity score may range from about 1 to 12. In some embodiments, a higher PDA score may indicate a higher risk for chronic lung disease. In some embodiments, a higher PDA scores may be greater than or equal to 5.5. In some embodiments, the threshold PDA severity score may be about 5.5. In some embodiments, the threshold PDA severity score may be associated with a sensitivity of about 94%. In some embodiments, the threshold PDA severity score may be associated with a specificity of about 93%. In some embodiments, the threshold PDA severity score may associated with a positive predictive value of about 94% and a negative predictive value of about 93%.

In another aspect, the current disclosure is directed to a method of treating Patent Ductus Arteriosus (PDA) in a subject comprising diagnosing hemodynamically significant PDA at one week after birth; and treating PDA in the subject comprising administering an effective amount of a pharmacological agent or performing a surgical procedure.

In some embodiments, diagnosing hemodynamically significant PDA may comprise determining a shunt volume across PDA. In some embodiments, diagnosing hemodynamically significant PDA may comprise measuring at least one surrogate of pulmonary over circulation; measuring at least one surrogate of systemic under perfusion, applying a model with one or more variable, wherein the one or more variable includes the at least one surrogate of pulmonary over circulation and the at least one surrogate of systemic under perfusion, generating an output value; and treating hemodynamically significant PDA comprising administering an effective amount of a pharmaceutical agent or performing a surgical procedure.

In some embodiments, the output value may be above a threshold value. In some embodiments, the threshold value may be about 5.5. In some embodiments, the one or more variable may further comprise an echocardiographic variable. In some embodiments, the model is a regression model comprising:

PDAss=(Gestational Age×−0.359)+(PPI in L/min/m²×0.418)+(LVO in mL/kg/min×0.001)+(SMA VTI in cm×−0.041)+(PV Vd in m/s×0.434)+(DFR in %×0.150)+11 (constant)

wherein the PDAss is the output variable, PPI is a pulmonary perfusion index measurement, LVO is a left ventricle measurement, SMA VTI is a superior mesenteric artery velocity time integral, PV Vd is a pulmonary vein diastolic flow measurement, and DFR is a reversal of flow in diastole in descending aorta measurement of the subject.

Another aspect of the present disclosure is directed to a method of predicting the risk of Patent Ductus Arteriosus (PDA) in a subject comprising identifying a gestational age of the subject; measuring one or more echocardiographic variables of the subject at one week after birth; determining a PDA severity score (PDAss) using a regression model; and calculating a percent risk of developing PDA in the subject; wherein the severity score is above a threshold PDA severity score.

In some embodiments, the echocardiographic variable measurement may be pulmonary perfusion index. In some embodiments, the echocardiographic variable measurement may be a left ventricle output. In some embodiments, the echocardiographic variable measurement may be a superior mesenteric artery velocity time integral. In some embodiments, the echocardiographic variable measurement may be a pulmonary vein diastolic flow. In some embodiments, the echocardiographic variable measurement may be a reversal of flow in diastole in descending aorta. In some embodiments, the subject may comprise a neonate having a gestational age of less than about 32 weeks. In some embodiments, the severity score may be about 5.5.

### BRIEF DESCRIPTION OF THE DRAWINGS

These and other features, aspects, and advantages of the present disclosure will become better understood when the following detailed description is read with reference to the accompanying drawings, in which like characters represent like parts throughout the drawings.

FIG. 1 is a graph showing a comparison of pulmonary perfusion index (PPI) in infants with and without CLD/death;

FIG. 2 is a graph showing a comparison patent ductus arteriosus (PDA) diameter in infants with and without CLD/death;

FIG. 3 is a graph showing a comparison of patent ductus arteriosus (PDA) maximum velocity (VMax) in infants with and without CLD/death;

FIG. 4 is a graph showing a comparison of left ventricle output (LVO) in infants with and without CLD/death;

FIG. 5 is a graph showing a comparison of LV a′ with and without CLD/death;

FIG. 6 is a graph showing a comparison of mitral valve E/A ratio with and without CLD/death;

FIG. 7 is a graph showing a comparison of pulmonary vein diastolic flow velocity with and without CLD/death;

FIG. 8 is a graph showing a comparison of diastole flow reversal in descending aorta with and without CLD/death;

FIG. 9 is a graph showing a comparison of SMA VTI with and without CLD/death;

FIG. 10 is a graph showing a comparison of celiac artery VTI aorta with and without CLD/death;

FIG. 11 is a graph showing a comparison of LA:Ao ratio with and without CLD/death;

FIG. 12 is a graph showing a difference in a PDA specificity score (PDAss) between infants with and without CLD/death; and

FIG. 13 is a graph showing the relationship between PDAss and predicted probability of CLD/death of the entire cohort.

### DETAILED DESCRIPTION

In describing and claiming the methods, the following terminology will be used in accordance with the definitions set forth below.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure may be described in the context of a single embodiment, the features may also be provided separately or in any suitable combination. Conversely, although the present disclosure may be described herein in the context of separate embodiments for clarity, the present disclosure may also be implemented in a single embodiment. The following definitions supplement those in the art and are directed to the current application and are not to be imputed to any related or unrelated case, e.g., to any commonly owned patent or application. Although any methods and materials similar or equivalent to those described herein may be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “some aspects,” “an embodiment,” “an aspect,” “another aspect”, “one embodiment,” “one aspect” or “other embodiments” or “other aspects” means that a particular feature, structure, or characteristic described in connection with the embodiments is included in at least some embodiments, but not necessarily all embodiments, of the present disclosures.

As used in this specification and the claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the disclosure, and vice versa.

The term “about” or “approximately” means within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. In another example, the amount “about 10” includes 10 and any amounts from 9 to 11. In yet another example, the term “about” in relation to a reference numerical value may also include a range of values plus or minus 10%, 9%, 8%, 7%, 6%, 5%, 4%, 3%, 2%, or 1% from that value. Alternatively, particularly with respect to biological systems or processes, the term “about” may mean within an order of magnitude, preferably within 5-fold, and more preferably within 2-fold, of a value. Where particular values are described in the application and claims, unless otherwise stated the term “about” meaning within an acceptable error range for the particular value should be assumed.

“Administering” is referred to herein as providing one or more compositions described herein to a patient or a subject. By way of example and not limitation, composition administration, e.g., injection, may be performed by intravenous (i.v.) injection, sub-cutaneous (s.c.) injection, intradermal (i.d.) injection, intraperitoneal (i.p.) injection, or intramuscular (i.m.) injection. One or more such routes may be employed. Parenteral administration may be, for example, by bolus injection or by gradual perfusion over time. Alternatively, or concurrently, administration may be by the oral route. Additionally, administration may also be by surgical deposition of a bolus or pellet of cells, or positioning of a medical device. In an embodiment, a composition of the present disclosure may comprise a vector comprising a nucleic acid sequence described herein, in an amount that is effective to treat, inhibit, or prevent infection. A pharmaceutical composition may comprise a vector as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The term “therapeutically effective amount”, therapeutic amount”, “immunologically effective amount”, or its grammatical equivalents refers to an amount effective, at dosages and for periods of time necessary, to achieve a desired therapeutic result. The therapeutically effective amount may vary according to factors such as the disease state, age, sex, and weight of the individual, and the ability of a composition described herein to elicit a desired response in one or more subjects. The precise amount of the compositions of the present disclosure to be administered may be determined by a physician with consideration of individual differences in age, weight, tumor size, extent of inflammation or disease, infection or metastasis, and condition of the patient (subject).

As used herein, the term “treatment”, “treating”, or its grammatical equivalents refers to obtaining a desired pharmacologic and/or physiologic effect. In some embodiments, the effect is therapeutic, i.e., the effect partially or completely cures a disease and/or adverse symptom attributable to the disease. In some embodiments, the term “treating” may include inhibiting or “preventing” a disease or a condition, including infection.

“Patient” or “subject” as used herein refers to a mammalian subject diagnosed with or suspected of having or developing an inflammatory or autoimmune disease. In some embodiments, the term “patient” refers to a mammalian subject with a higher than average likelihood of developing an inflammatory disorder. Exemplary patients may be humans, apes, dogs, pigs, cattle, cats, horses, goats, sheep, rodents, and other mammalians that may benefit from the therapies disclosed herein. Exemplary human patients may be male and/or female. “Patient in need thereof” or “subject in need thereof” is referred to herein as a patient diagnosed with or suspected of having a disease or disorder, for instance, but not restricted to PDA. In some embodiments, patients may be neonatal patients that are infants of less than one week old.

As used herein, the term “surrogate” is intended to encompass a marker or an indicator to describe any medical event of condition. For example, echocardiographic variables may be surrogates or indicators of pulmonary over circulation and systemic under perfusion.

As used herein, the term “hemodynamically significant PDA” refers to the evaluation of several factors: (1) PDA shunt volume assessment and its impact on the systemic and pulmonary circulations; (2) myocardial function evaluation, especially in considering how the heart handles the increased preload in the setting of potential myocardial ischemia secondary to impaired coronary artery perfusion; (3) antenatal and perinatal characteristics that can act as effect modifiers to either mitigate or exacerbate potential detrimental consequences of a shunt. Defining hemodynamic significance thus requires a thorough appraisal of all of these factors.

As used herein, the term “gestational age” is intended to describe the age of an infant from the last menstrual period of the mother until birth.

As used herein, the term “echocardiographic variable” includes measurements to determine heart and lung function. For example, echocardiographic variable may be measured by an electrocardiograph, including, but not limited to pulmonary perfusion index (PPI), left ventricle output (LVO), smooth mesenteric artery velocity time integral (SMA VTI), peak diastolic flow velocity (PV Vd), and diastolic flow reversal in the descending aorta (DFR).

As used herein, the term “outcome” includes diseases associated with a condition being experienced by a patient. For example, the PDA severity score described herein corresponds to chronic lung disease and/or death.

As used herein, the term “shunt volume” refers to the size of the open communication (or diameter) and the pulmonary vascular resistance (PVR).

As used herein, the term “periventricular leukomalacia” is a type of brain injury in premature babies and may refer to evidence of white matter degeneration noted on cranial ultrasound or magnetic resonance imaging.

As used herein, the term “intraventricular hemorrhage (Grade III or IV)” refers to the diagnosis of a hemorrhage by cranial ultrasound using Papile classification system.

As used herein, the term “chronic lung disease” includes a disease of the lung and other parts of the respiratory system. Chronic lung disease may include a requirement of oxygen at 25 weeks, 26 weeks, 27 weeks, 28 weeks, 29 weeks, 30 weeks, 31 weeks, 32 weeks, 33 weeks, 34 weeks, 35 weeks, or 36 weeks of gestation.

The present disclosure is directed to utilizing echocardiography for assessing PDA hemodynamic significance to supplement a patient's clinical and laboratory data at early time points after birth. The present disclosure is also directed to methods for assessing flow in branched pulmonary arteries as a measure of detecting hemodynamically significant PDA (hsPDA) and predicting a primary composite outcome of CLD or death. Thus in one embodiment described herein is method of treating Patent Ductus Arteriosus (PDA) in a subject comprising: identifying a clinical variable of the subject; measuring one or more echocardiographic variables of the subject at one week after birth; determining a PDA severity score using a regression model; and treating PDA in the subject comprising administering an effective amount of a pharmacological agent or performing a surgical procedure; wherein the severity score is above a threshold PDA severity score.

A heterogenic set of variables associated with PDA must be identified and evaluated to determine if they have prognostic significance, and are informative in making a PDA diagnosis. These include, but are not limited to, echocardiographic and/or clinical variables. Electrocardiographic variable are measurements used to show changes in the cardiovascular and pulmonary activity over time, and may include, but are not limited to, a level of systemic perfusion, a level of pulmonary perfusion, a left ventricular LV function, a pulmonary perfusion index, a left ventricle output, a superior mesenteric artery velocity time integral, a pulmonary vein diastolic flow, and a reversal of flow in diastole in the descending aorta. Level of systemic perfusion refers to the total amount of blood flowing through the circulatory system of the patient and the level of pulmonary perfusion refers to a total volume of blood reaching the pulmonary capillaries of the patient.

Echocardiographic variables obtained within one week of birth of 98 patients (or 124 confirm) were used to develop a predictive model since early measurements of echocardiographic variables may be useful in decision making regarding future treatment. Additionally, measurements made after one week may reflect the clinical effects of a significant PDA shunt, which usually occur after the first week of life. The echocardiographic variables may be suggestive of either pulmonary over perfusion or systemic hypo perfusion, and measurements of left ventricle (LV) diastolic function are included in the predictive model to derive a PDA specificity score (PDAss). Thus in one aspect, electrocardiographic variable comprises a level of systemic perfusion, a level of pulmonary perfusion, or a left ventricular LV function.

In contrast, clinical variables comprise various clinical attributes that may typically be available at presentation. Clinical variables include, but are not limited to, gestational age. Gestational age (in the format of weeks/days) may be used as a clinical variable in the predictive model. Other cardiorespiratory characteristics, such ase ventilator settings, blood pressure, and oxygen requirement, may be used as clinical variables. Thus, in another aspect described herein, cardiorespiratory characteristics, including but not limited to, ventilator settings, blood pressure, and oxygen requirements, are highly correlated with gestational age and may not be considered for devising the severity score. In another aspect, the clinical variables comprise gestational age. Gestational age of a patient may be measured in weeks, from the first day of the woman's last menstrual cycle to the date of delivery. A normal pregnancy may range from 38 to 42 weeks. Infants born before 37 weeks may be considered premature. Thus gestational age may comprise weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, or 40 weeks. Once identified, the selected variables serve as inputs into a predictive regression model used to determine the PDAss. In another aspect, the clinical variables may comprise cardiorespiratory characteristics such as ventilator settings, blood pressure, and/or oxygen requirements. In a further aspect, clinical variables may not comprise cardiorespiratory characteristics like ventilator settings, blood pressure, and/or oxygen requirement that are highly correlated with gestational age.

The PDAss may be used for diagnosing hemodynamically significant PDA (hsPDA) based on the occurrence of a primary outcome. The primary outcome is a patient condition indicative of hemodynamically significant PDA (hsPDA). In one aspect, the predictive regression model may be applied for deriving a PDAss for predicting the primary outcome of the patient. In one aspect, the primary outcome of the patient may comprise chronic lung disease (CLD) or death. Although, various clinical outcomes may be considered as primary outcomes, including necrotizing enterocolitis (NEC), intraventricular hemorrhage (IVH), invasive ventilation and sepsis, in some aspects, these are not used in determining the PDAss because they are diagnosed after the first week of life, and thus may not have early predictive ability. Additionally, in one aspect, a composite primary outcome comprising CLD and death may be used even though CLD and death may be mutually exclusive outcomes.

The occurrence of the primary outcome may thus depend on the value of the PDAss determined by a predictive regression model. In one aspect, the predictive regression model is a multivariate logistic regression model, which calculates the possibility of the primary outcome based on multiple variables (e.g., the clinical and electrographic variables). The model may thus include five electrocardiographic variables and one clinical variable. Furthermore, a β coefficients of each echocardiographic variable may be used to signify the amount change by which that specific echocardiographic variables must be multiplied to give the corresponding average change in the PDAss. Thus another aspect described herein, is a weighted scoring system based on a β coefficient of the echocardiographic variables that are determined to be predictive of the primary outcome and may be used in the predictive regression model to derive the PDAss.

In accordance with one aspect, five echocardiography variables include in the predictive model comprise pulmonary perfusion index (PPI) (L/min/m²), left ventricle output (LVO) (mL/kg/min), smooth mesenteric artery velocity time integral (SMA VTI) (cm), peak diastolic flow velocity in the pulmonary vein (PV Vd) (m/s), diastolic flow reversal in the descending aorta (DFR) (%)]. The perfusion index (PI) is the ratio of the pulsatile blood flow to the static blood in the patient's peripheral tissue. Left ventricular (LV) output is a measure of stroke volume output per minute. Smooth mesenteric artery velocity time integral (SMA VTI) is a clinical measurement of blood flow in the smooth mesenteric artery, equivalent to the area under the velocity time curve. Peak diastolic velocity in the pulmonary vein (PV) is the velocity of blood flow in the pulmonary vein during peak diastole. Diastolic flow reversal in the descending aorta (DFR) is measure of change in direction of blood flow in the descending aorta.

Table 1 demonstrates the unstandardized β coefficients of the five echocardiography variables and the one clinical variable which denote the relative importance and significance of each independent variable.

TABLE 1
Regression model used to derive PDAss.

| Variable | Unstandardized β | Standardized β | P value |
|---|---|---|---|
| Gestational Age | −0.359 | −0.086 | <0.01 |
| PPI (L/min/m²) | .418 | .506 | <0.01 |
| LVO (mL/kg/min) | .001 | .187 | .01 |
| SMA VTI (cm) | −.041 | −.152 | .02 |
| PV Vd (m/s) | .434 | .151 | .02 |
| DFR (%) | .150 | .298 | .01 |

Thus, in another aspect, the following equation (Equation 1) based on the regression model may be used to calculate the PDAss for each patient:

PDAss=(Gestational Age×−0.359)+(PPI in L/min/m²×0.418)+(LVO in mL/kg/min×0.001)+(SMA VTI in cm×−0.041)+(PV Vd in m/s×0.434)+(DFR in %×0.150)+11 (constant) (1)

In another aspect, the PDAss may range from 0 (low risk) to 12 (highest risk). The predicted probability for each patient to develop a certain primary outcome, such as, chronic lung disease and/or death may be based on the PDAss derived from the model identified in Equation 1. A threshold PDAss may be the PDAss score that is the minimum required for treatment. In one aspect, the threshold PDAss triggering treatment for PDA is 5.5. In another aspect, the PDAss has a sensitivity of 99%, 98%, 97%, 96%, 95%, 94%, 93%, 92%, 91%, or 90%. In one aspect, the PDAss of 5.5 has a sensitivity of 94%, a specificity of 93%, a positive predictive value of 94% and a negative predictive value of 93%. The negative and positive predictive values of the threshold may be relatively high, implying the generalizability to other populations. Hence, PDAss can be used with this threshold to differentiate severity in further prospective studies for external validation and also have a potential use in randomized clinical trials to determine selection for early treatment. In one aspect, the method may also include directly assessing flow across the PDA into pulmonary arteries to predict a primary outcome with reasonable accuracy.

Another embodiment described herein is a method of treating PDA in a patient in need thereof, comprising diagnosing hemodynamically significant PDA (hsPDA) after birth. The method comprises measuring at least one surrogate of pulmonary over circulation, measuring at least one surrogate of systemic under perfusion, applying a model with one or more variable, generating an output value, and treating hemodynamically significant PDA comprising administering an effective amount of a pharmaceutical agent or performing a surgical procedure.

In one aspect, the method may comprise treating PDA in a patient comprising diagnosing hemodynamically significant PDA (hsPDA) at one to seven days after birth, at one to three weeks after birth, or at three to five weeks after birth, including any time period or range comprised therein. In one aspect, the surrogate of the pulmonary over circulation may be a marker or indicator of over circulation in the patient. In another aspect, the surrogate of the systemic under perfusion may be a marker or indicator of systemic under perfusion in the patient. In another aspect, the surrogate of pulmonary over circulation and/or the surrogate of systemic under perfusion may include one or more echocardiographic variables including, but not limited Left Pulmonary Artery (LPA) diameter (mm), LPA systolic and diastolic velocity time integral (VTI) in cm, LPA flow (L/min/m²), right pulmonary artery (RPA) diameter (mm), RPA systolic and diastolic VTI (cm), RPA flow (L/min/m²), PDA diameter (mm), PDA velocity (m/s), mitral inflow velocities E wave (m/s), A wave (m/s) and E/A ratio, peak diastolic flow velocity (PV Vd) in pulmonary vein (m/s), diastolic flow reversal in the descending aorta (DFR) (%), systolic and diastolic flow VTI in smooth mesenteric artery and celiac artery (cm), left ventricle output (LVO in mL/kg/min), ratio of left atrial size to aortic root diameter (LA:Ao ratio).

In one aspect, the output value may be the PDAss determined based on equation 1 described above. In another aspect, treating hemodynamically significant PDA in the patient may comprise administering an effective amount of a pharmaceutical agent including, but not limited to non-steroidal anti-inflammatory drugs (e.g., indomethacin, ibuprofen), naproxen, diclofenac, celecoxib, acetaminophen and paracetamol. Alternatively or additionally, the patient may be surgically treated with interventions including, but not limited to cardiac catheterization, trans-catheter closure and surgical ligation.

Another embodiment described herein is a method of predicting risk of PDA in a patient. The method comprises devising a risk score based on clinical and echocardiographic variables of the patient. The unstandardized β coefficients may be used to devise the risk score. Negative β coefficients indicate that higher variable values are associated with a decrease in the risk of developing the outcome. Positive β coefficients indicate that higher variable values are associated with an increase in the risk of developing the disease. In one aspect, equation 1 described above may be used to calculate the risk factor each patient.

In accordance with one aspect, the derived PDAss may be utilized in predicting clinical outcomes of CLD or death in patients less than about 32 weeks. The relative influence of each variable may be inferred from Table 1 based on the unstandardized (3 coefficients. Pulmonary vein diastolic velocity and PPI are determined to have the biggest influence whereas left ventricle output and smooth mesenteric artery velocity time integral velocity time integral are determined to have relatively lower significance. This may be explained by the fact that surrogates of pulmonary over circulation play a more important role in development of CLD rather than surrogates of systemic under perfusion. Unexpectedly, PDA diameter was not significantly different in groups that had the primary outcome of CLD/death and the groups that did not have that primary outcome. Thus, PDA diameter was not included in the model. This signifies that pulmonary overflow is determined by the pressure difference across the duct and not by duct size.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

### Example 1: Design of Study

One hundred and twenty four patients with PDA detected during the first week of life were enrolled during the study period and 98 infants met inclusion criteria (pulmonary hypertension [n=20], major congenital anomalies [n=3], inadequate data [n=3]). These 98 infants were considered patients. The mean gestational age and birth weight of the study cohort was 28.9±1.91 weeks and 1228.06±318.94 grams, respectively. Sixty four patients met criteria for the primary outcome of CLD or death (death [n=2]). The cause of death was severe respiratory failure in both patients, one of which also had culture proven gram negative sepsis.

Table 2 demonstrates the comparison of demographic information, perinatal data, and clinical characteristics among both the groups. Patients with the primary outcome of CLD or death had significantly lower gestation age and birth weight. Additionally, there was a higher incidence of pre-eclampsia and a comparatively lower incidence of administration of antenatal steroids to the mother before delivery in the CLD/death group. Among clinical characteristics, lower pH, slightly higher mean airway pressure, upper range of fraction of inspired oxygen, marginally lower blood pressure were noted in the CLD/death group. Although statistically significant, the clinical relevance of these differences was low.

Table 3 illustrates the differences in other clinically relevant outcomes amongst both the groups. Longer days of invasive ventilation, increased use of postnatal steroids, furosemide, and inotropes were notable in the CLD/death group. Incidence of severe intraventricular hemorrhage and culture positive sepsis was higher in the CLD/death group.

Other outcomes including necrotizing enterocolitis, PVL, and pulmonary hemorrhage were compared between the groups. Calculations were not computed due to the low prevalence and statistical insignificance.

TABLE 2
Demographic characteristics and clinical details among both groups.

| Characteristics | CLD/death (n = 34) | | No CLD/death (n = 64) | | p value |
|---|---|---|---|---|---|
| Gestation (wk) | 27.39 ± 1.44 | | 29.74 ± 1.61 | | <0.001 |
| Birth weight (g) | 1038.65 ± 215.02 | | 1317 ± 327.20 | | <0.001 |
| Male | 22 | (64%) | 37 | (58%) | 0.33 |
| Vaginal delivery | 21 | (62%) | 36 | (56%) | 0.42 |
| Preeclampsia | 9 | (26%) | 9 | (14%) | 0.04 |
| Use of MgSO4 | 23 | (68%) | 40 | (63%) | 0.67 |
| Chorioamnionitis | 3 | (9%) | 5 | (8%) | 0.91 |
| Cord pH | 7.31 ± 0.12 | | 7.33 ± 0.16 | | 0.82 |
| Antenatal Steroids | | | | | 0.04 |
| None | 4 | (12%) | 9 | (14%) | |
| 1 dose | 8 | (24%) | 19 | (30%) | |
| 2 doses | 22 | (64%) | 36 | (56%) | |
| Apgar score at 5 min | 8 | [6-9] | 9 | [7-9] | 0.16 |
| Clinical characteristics* | | | | | |
| pH | 7.29 ± 0.11 | | 7.31 ± 0.14 | | 0.03 |
| Mechanical ventilation | 19 | (56%) | 27 | (42%) | <0.001 |
| Mean airway pressure | 8 ± 2 | | 7 ± 2 | | 0.05 |
| (mmHg) | | | | | |
| Inspired oxygen fraction † | 21 | [21-67] | 21 | [21-44] | <0.001 |
| (%) | | | | | |
| Oxygen saturation (%) | 95 ± 3 | | 97 ± 2 | | 0.03 |
| Total fluid intake | 110 | [100-130] | 100 | [90-120] | 0.15 |
| (mL/kg/day) | | | | | |
| Systolic blood pressure | 55 | [50-60] | 57 | [51-65] | 0.04 |
| (mmHg) | | | | | |
| Diastolic blood pressure | 29 | [22-36] | 32 | [27-38] | 0.02 |
| (mmHg) | | | | | |

MgSO4—Magnesium sulphate
Values are presented as n (%) and Mean ± SD or Median [IQR] as appropriate
† Presented as median [range].
*Clinical characteristics displayed were recorded at the time of the echocardiogram

TABLE 3
Distribution of clinical outcomes and interventions among both groups.

| | CLD/death before | | No CLD/death before | | |
|---|---|---|---|---|---|
| | discharge | | Discharge | | p value |
| NEC | 5 | (15%) | 3 | (5%) | 0.07 |
| Clinical suspicion | 2 | | 2 | | |
| Medical therapy | 2 | | 1 | | |
| Surgical therapy | 1 | | 0 | | |
| Days of mechanical ventilation | 18 | [6-34] | 2 | [0-5] | <0.001 |
| Pulmonary hemorrhage | 1 | | 0 | | — |
| Furosemide | 17 | | 12 | | <0.001 |
| Inotropes | 14 | | 7 | | <0.001 |
| Postnatal steroids | 12 | | 2 | | <0.001 |
| Culture-positive sepsis | 3 | | 1 | | <0.001 |
| IVH (Grade 3 or 4) | 2 | | 1 | | 0.004 |
| PVL | 1 | | 0 | | — |
| PDA treatment (after 1 week of life) | 20 | (58%) | 12 | (19%) | <0.001 |
| Ibuprofen | 20 | (58%) | 12 | (19%) | <0.001 |
| Paracetamol | 8 | (24%) | 4 | (6%) | <0.001 |

| | | | | |
|---|---|---|---|---|
| Trans catheter Closure | 3 | (9%) | 0 | — |
| Surgical Ligation | 1 | (3%) | 0 | — |

### Example 2: Identification of Echocardiographic Variables for the Model

Collinearity diagnostics was done to identify echocardiographic variables that were highly correlated with each other (velocity time integral>2.5). Collinearity diagnostics helps in identifying variables that may be correlated and that may not independently predict PDAss. A p value >0.05 was considered non-predictive and those echocardiographic variables were removed from the predictive model. For example, markers for left ventricle diastolic function were assessed as important when there is increased blood volume on the left side of the heart (increased preload). However, due to high collinearity with other variables, markers for left ventricle diastolic function were not included in the final model. Similarly, celiac artery velocity time integral, a marker of systemic perfusion, was also not included in the predictive model due to high collinearity with other markers of systemic under perfusion. The five echocardiography variables and one clinical variable in the predictive regressive model identified in equation were tested for collinearity. Variance inflation factor for all six variables was found to be less than 1.5.

### Example 3: Collection of Echocardiographic Data

The median (IQR) time period of performance of the echocardiography was 54 hours (39-70). The scan was performed using the Epiq (Philips Medical Systems, Andover, MA, USA) by an experienced sonographer using standardized protocol in accordance with recent guidelines published by the American Society of Echocardiography. All images were digitally stored for further analysis and the additional measurements were extracted from the offline archive using Merge Cardio Software (IBM Watson Health, Cambridge, MA, USA) which serves as a secure storage platform for performed echocardiograms. Care was taken to rule out any congenital heart disease other than a PDA or a patent foramen ovale (PFO), and if present, immediately communicated to the attending neonatologist.

Echocardiographic variables included surrogates or indicators of PDA characteristics, pulmonary over circulation and systemic under perfusion, and LV function: Left Pulmonary Artery (LPA) diameter (mm), LPA systolic and diastolic velocity time integral (VTI) in cm, LPA flow (L/min/m²), right pulmonary artery (RPA) diameter (mm), RPA systolic and diastolic VTI (cm), RPA flow (L/min/m²), PDA diameter (mm), PDA velocity (m/s), mitral inflow velocities E wave (m/s), A wave (m/s) and E/A ratio, peak diastolic flow velocity (PV Vd) in pulmonary vein (m/s), diastolic flow reversal in the descending aorta (DFR) (%), systolic and diastolic flow VTI in smooth mesenteric artery and celiac artery (cm), left ventricle output (LVO in mL/kg/min), ratio of left atrial size to aortic root diameter (LA:Ao ratio).

The narrowest PDA diameter was measured using 2-dimensional echocardiogram at the narrowest part, which is the pulmonary end. The maximum shunt velocity and flow across the PDA, LPA and RPA [Vmax] was measured with highest Nyquist limit needed for laminar flow. Tissue Doppler imaging (TDI) of the apical 4-chamber view was used for LV early diastolic (e′), and late diastolic (a′) velocities (m/s) measured using a pulsed wave Doppler at the level of the lateral mitral valve annulus. If the e′ and a′ waves were fused, the visualized single wave was measured as the a′ wave. Flow (L/min/m²) in the pulmonary arteries was measured as follows: the branched pulmonary artery diameter (d) was measured 5 mm from the level of the bifurcation from the Main Pulmonary Artery (MPA) using the short parasternal axis view. Cross sectional area (CSA) of the branched pulmonary arteries were calculated from the diameter. The velocity time integral of LPA and RPA were measured using the pulsed wave Doppler in the same view. The Doppler gate cursor was aligned to make sure it is parallel to the direction of flow in the branches. Angle correction was not used. An average of 3 simultaneous Doppler wave forms were captured to be utilized for calculation of velocity time integral. Pulmonary perfusion index (PPI) measuring flow in each of the pulmonary artery branches was then calculated using the following equation and then averaged to give a single value:

Pulmonary Perfusion Index(L/min/m²)=(CSO ×VTI ×heart rate)÷Body Surface Area (2)

All measurements were performed by an experienced cardiac sonographer blinded to the clinical data. The primary outcome of the study was a composite of CLD/death before discharge. Based on the ability of the El-Khuffash score to predict CLD/death, it was estimated that the score had at least a 92% area under the curve with 95% confidence level and a confidence interval of 0.125 with 30% combined prevalence of the composite primary outcome, the expected total sample size was 88 (Hanley, 1982).

### Example 3: Statistical Analysis

The cohort was divided into two groups based on the presence of primary composite outcome of CLD/death. Categorical variables were expressed as n (%) and continuous variables as mean (SD) or median (IQR) when appropriate. Categorical and continuous variables will be compared by χ²/Fischer exact and Student t test/Mann Whitney test as appropriate, respectively after testing for normality using the Shapiro-Wilk test. Univariate analysis was performed to identify the echocardiographic variables associated with primary outcome. A P value of <0.05 was considered to be statistically significant. All statistical analysis were performed using IBM SPSS Statistics for Macintosh, v26.0 (IBM Corp, Armonk, New York)

### Example 4: Validating the Predictive Regression Model

The predictive accuracy of PDAss determined using equation 1 was compared to that of the predictive accuracy of using PDA diameter, LA:Ao ratio and DFR independently.

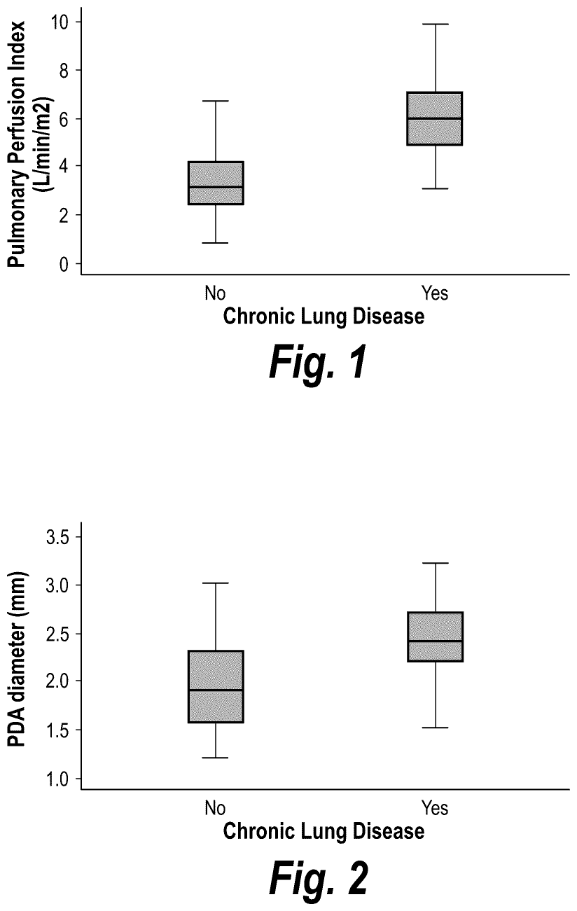

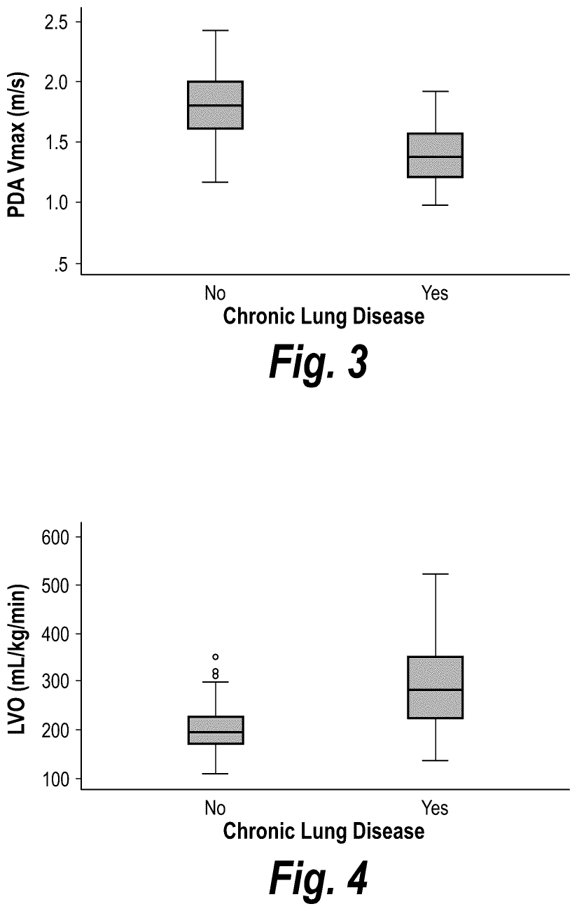

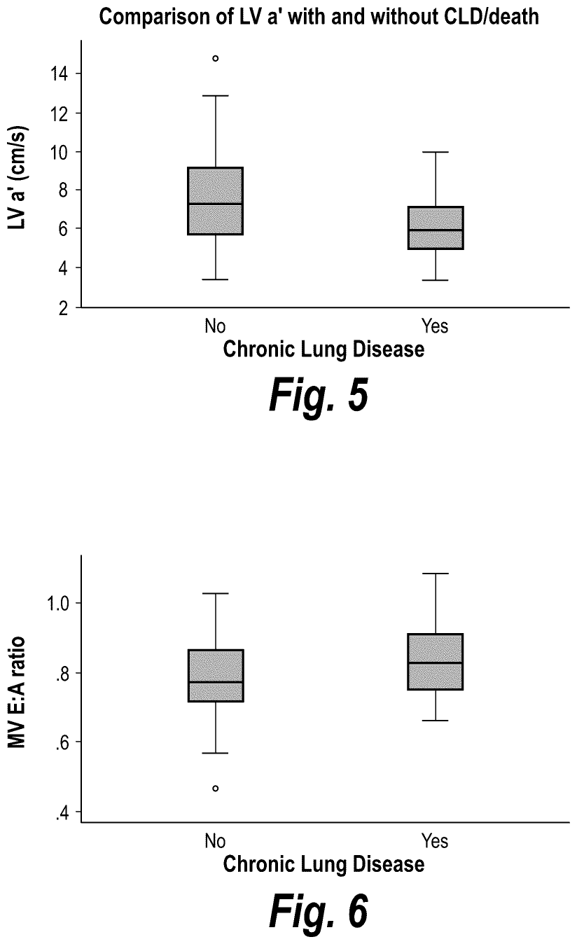

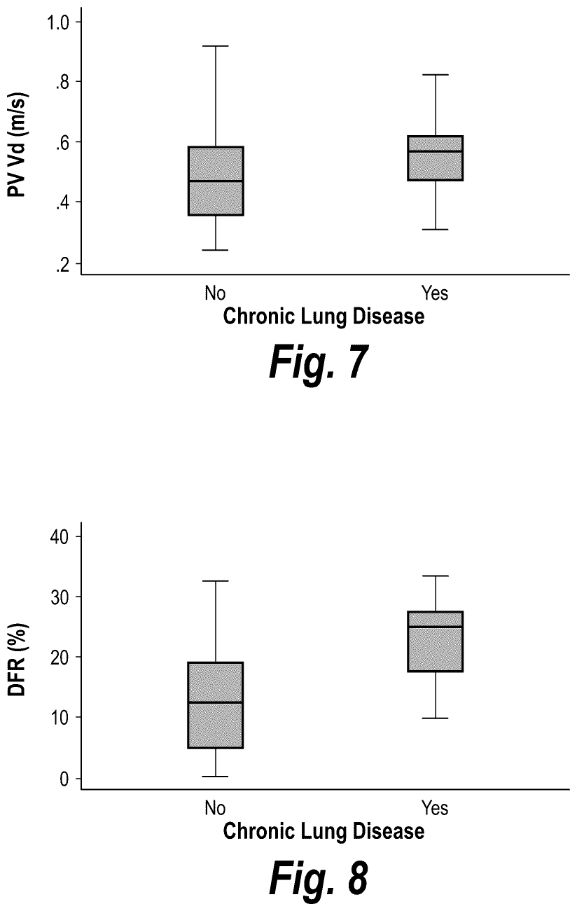

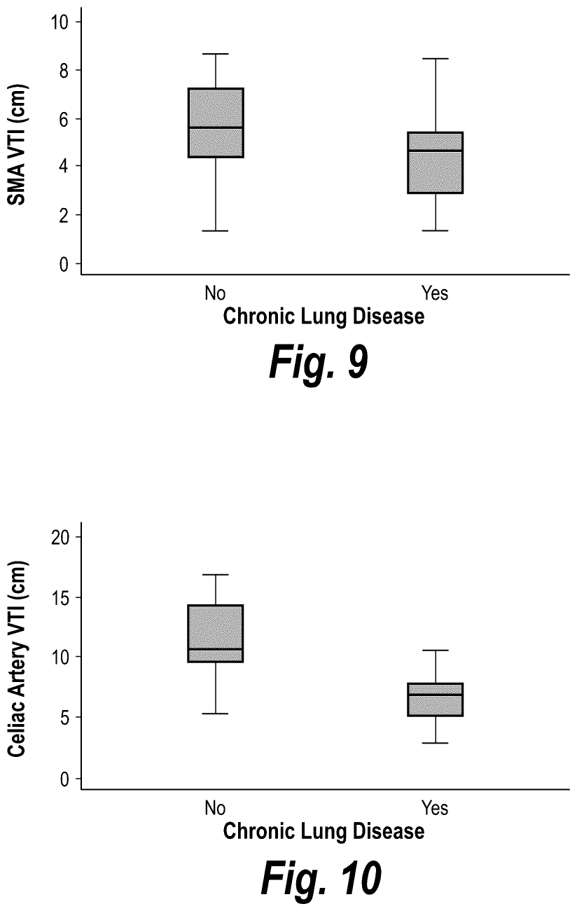

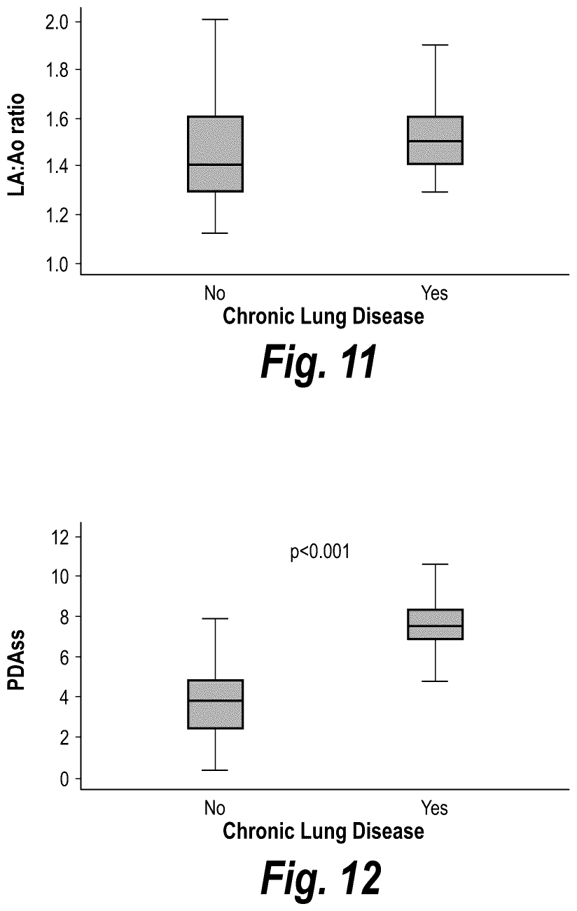

In accordance with one aspect, the ability of each echocardiography variable to predict the primary outcome of chronic lung disease or death was individually assessed in a cohort of 98 patients. FIGS. 1-11 illustrate the impact of echocardiographic variables on the two groups. PPI was significantly higher in the CLD/death group (p<0.001). Similarly, LV output (p=0.01), pulmonary vein diastolic flow velocity (p=0.05), SMA VTI (p=0.03), and diastolic flow reversal in the descending aorta (p=0.01) were all significantly higher for patients in the CLD/death group. PDA diameter, PDA maximum velocity (Vmax), LV a′ measured by TDI, LA:Ao ratio and mitral valve E/A ratio were not statistically significant between the two groups (CLD/death group and no CLD/death group).

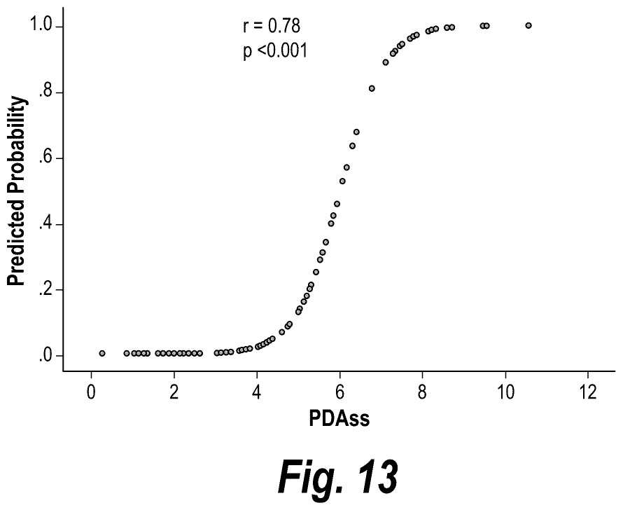

The mean risk score of the entire cohort was 4.99±2.35, while the patients in the CLD/death group had a higher score (7.5 [1.2] vs 3.6 [1.5], p<0.001) compared to those who were not in the CLD/death group (see FIG. 12). Thus, a strong correlation was demonstrated between the predicted probability of developing the primary outcome (CLD/death) which was derived using the model and the PDAss (see FIG. 13).

In accordance with one embodiment, a receiver operating characteristic curve was constructed to probe the predictive ability of the severity score to predict CLD/death in the cohort. PDAss yielded an area under the curve (AUC) of 0.97 (95% CI 0.93-0.99, p<0.001) for predicting CLD/death. A receiver operating characteristic (ROC) curve is defined as a plot of test sensitivity versus specificity or false positive rate (FPR) for a given variable. When compared with individual parameters including PDA diameter (AUC), LA:Ao ratio (AUC) and DFR (AUC) which are traditionally used to demonstrate hemodynamic significance, the predictive model described in this disclosure fared better (FIG. 4).

The relationship between the PDAss and the primary outcome was further probed by controlling for other clinical variables listed in Table 1 that were associated with CLD/death on univariate analysis. After controlling for severe IVH, sepsis, postnatal steroid use, furosemide use, inotrope support, PDAss remained significant [aOR 2.76 (95% CI 1.89-3.52), p<0.001]. Finally, PDAss was compared between patients with and without NEC showing a higher score in those with as compared to those without NEC (6.5±2.7 vs 3.8±1.7, p=0.03). The AUC for NEC predictive ability of the score was 0.76 (95% CI [0.59-0.94], p=0.02).

1. A method of treating Patent Ductus Arteriosus (PDA) in a subject comprising:
identifying a clinical variable of the subject;
measuring one or more echocardiographic variables of the subject one week after birth;
determining a PDA severity score using a regression model; and
treating PDA in the subject comprising administering an effective amount of a pharmacological agent or performing a surgical procedure;
wherein the severity score is above a threshold PDA severity score (PDAss).

2. The method of claim 1, wherein the clinical variable and echocardiographic variable is a measurement associated with PDA in the subject.

3. The method of claim 1, wherein the clinical variable comprises a gestational age.

4. The method of claim 2, wherein the echocardiographic variable measurement comprises a level of systemic perfusion, a level of pulmonary perfusion, or a left ventricular LV function.

5. The method of claim 2, wherein the echocardiographic variable measurement is a pulmonary perfusion index.

6. The method of claim 2, wherein the echocardiographic variable measurement is a left ventricle output.

7. The method of claim 2, wherein the echocardiographic variable measurement is a superior mesenteric artery velocity time integral.

8. The method of claim 2, wherein the echocardiographic variable measurement is a pulmonary vein diastolic flow.

9. The method of claim 2, wherein the echocardiographic variable measurement is a reversal of flow in diastole in descending aorta.

10. The method of claim 1, wherein the regression model is:
PDAss=(Gestational Age×−0.359)+(PPI in L/min/m²×0.418)+(LVO in mL/kg/min×0.001)+(SMA VTI in cm×−0.041)+(PV Vd in m/s×0.434)+(DFR in %×0.150)+11 (constant)
wherein PPI is a pulmonary perfusion index measurement, LVO is a left ventricle measurement, SMA VTI is a superior mesenteric artery velocity time integral, PV Vd is a pulmonary vein diastolic flow measurement, and DFR is a reversal of flow in diastole in descending aorta measurement of the subject.

11. The method of claim 1, wherein the PDA severity score ranges from about 1 to 12.

12. The method of claim 1, wherein a higher PDA score indicates a higher risk for chronic lung disease.

13. The method of claim 13, where in a higher PDA scores is greater than or equal to 5.5.

14. The method of claim 1, wherein the threshold PDA severity score is associated with a sensitivity of about 94%.

15. The method of claim 1, wherein the threshold PDA severity score is associated with a specificity of about 93%.

16. A method of treating Patent Ductus Arteriosus (PDA) in a subject comprising:
diagnosing hemodynamically significant PDA at one week after birth; and
treating PDA in the subject comprising administering an effective amount of a pharmacological agent or performing a surgical procedure.

17. The method of claim 16, wherein the diagnosing hemodynamically significant PDA comprises:
measuring at least one surrogate of pulmonary over circulation;
measuring at least one surrogate of systemic under perfusion,
applying a model with one or more variable, wherein the one or more variable includes the at least one surrogate of pulmonary over circulation and the at least one surrogate of systemic under perfusion,
generating an output value; and
treating hemodynamically significant PDA comprising administering an effective amount of a pharmaceutical agent or performing a surgical procedure.

18. A method of predicting the risk of Patent Ductus Arteriosus (PDA) in a subject comprising:
identifying a gestational age of the subject;
measuring one or more echocardiographic variables of the subject at one week after birth;
determining a PDA severity score (PDAss) using a regression model; and
calculating a percent risk of developing PDA in the subject;
wherein the severity score is above a threshold PDA severity score.

19. The method of claim 18, wherein the subject comprises a neonate having a gestational age of less than about 32 weeks.

20. The method of claim 18, wherein the regression model is:
PDAss=(Gestational Age×−0.359)+(PPI in L/min/m²×0.418)+(LVO in mL/kg/min×0.001)+(SMA VTI in cm×−0.041)+(PV Vd in m/s×0.434)+(DFR in %×0.150)+11 (constant)
wherein PPI is a pulmonary perfusion index measurement, LVO is a left ventricle measurement, SMA VTI is a superior mesenteric artery velocity time integral, PV Vd is a pulmonary vein diastolic flow measurement, and DFR is a reversal of flow in diastole in descending aorta measurement of the subject.
